(19) United States
(12) Patent Application Publication
(10) Pub. No.: US 2010/0081809 A1
(43) Pub. Date: Apr. 1, 2010

(54) AMORPHOUS VALGANCICLOVIR HYDROCHLORIDE

(75) Inventors: Surya Narayana Devarakonda, Hyderabad (IN); Sesha Reddy Yerraguntla, Hyderabad (IN); Venu Nalivela, Warangal (IN); Arjun Kumar Tummala, Guntur (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LIMITED, Hyderabad (IN); DR. REDDY'S LABORATORIES, INC., Bridgewater, NJ (US)

(21) Appl. No.: 12/607,187

(22) Filed: Oct. 28, 2009

Related U.S. Application Data
(63) Continuation of application No. 12/204,949, filed on Sep. 5, 2008, now abandoned.
(60) Provisional application No. 61/054,062, filed on May 16, 2008.

(30) Foreign Application Priority Data
Sep. 5, 2007 (IN) 1996/CHE/2007

(51) Int. Cl. C07D 473/18

(52) U.S. Cl. 544/276

(57) ABSTRACT

The present application relates to amorphous forms of valganciclovir salts such as the hydrochloride and processes for their preparation.

### TECHNICAL FIELD

The present application relates to amorphous forms of valganciclovir salts such as the hydrochloride and processes for their preparation.

### BACKGROUND

Valganciclovir hydrochloride is the adopted name for the drug having chemical name L-valine, 2-[(2-amino-1, 6-dihydro-6-oxo-9H-purin-9-yl)-methoxy]-3-hydroxypropanyl ester, monohydrochloride and is represented by structural Formula I. Valganciclovir is a mono-L-valyl ester prodrug of the antiviral compound ganciclovir.

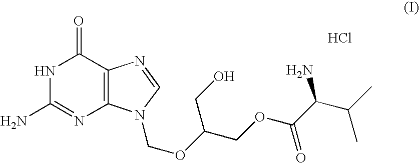

Valganciclovir hydrochloride is currently marketed in the U.S. under the brand name Valcyte® as a 450 mg tablet for oral administration. Valcyte® tablets are indicated for the treatment of cytomegalovirus (CMV) retinitis in patients with acquired immunodeficiency syndrome (AIDS). Valcyte® is indicated for the prevention of cytomegalovirus (CMV) disease in kidney, heart, and kidney-pancreas transplant patients at high risk (Donor CMV seropositive/Recipient CMV seronegative [(D+/R−)]).

There is a need for an amorphous form of valganciclovir hydrochloride, either alone or in combination with a pharmaceutically acceptable carrier.

There is a general need for solids of pharmaceutically active compounds that can be produced by processes giving products suitable for pharmaceutical preparations. It is desirable for such products to have the properties such as easy to handle, good dissolution rate, excellent bioavailability and good storage stability. Since polymorphic forms can vary in their physical properties, regulatory authorities require that efforts be made to identify all polymorphic forms, e.g., crystalline, amorphous, solvated etc. of new drug substances.

New forms of pharmaceutically useful compounds provide an opportunity to improve the performance characteristics of such products. Further, discovery of additional polymorphic forms may help in the identification of the polymorphic content of a batch of an active pharmaceutical ingredient. Therefore, there is a need for preparing new solid forms of a drug substance and processes for preparation thereof.

### SUMMARY

In one aspect, the present patent application provides essentially pure, substantially pure, and/or pure amorphous valganciclovir hydrochloride. These levels of purity relate to impurities such as unwanted solvents, reaction products and the like, and/or other solid forms such as crystalline forms. Also provided is a combination of amorphous valganciclovir hydrochloride with a pharmaceutically acceptable carrier. Processes for preparation of these solids are also provided.

In one embodiment, there is provided a process for preparation of substantially pure amorphous valganciclovir hydrochloride by removing a solvent from a solution, suspension or dispersion of valganciclovir hydrochloride.

In another embodiment, there is provided a process for preparation of combination of amorphous valganciclovir hydrochloride with a pharmaceutically acceptable carrier, which includes removing a solvent from a solution of valganciclovir hydrochloride and a suitable pharmaceutical carrier where one or both are dissolved or dispersed or suspended in an organic solvent.

In yet another embodiment, there is provided a pharmaceutical composition that includes amorphous valganciclovir or its pharmaceutically acceptable salt in a free drug particulate form and one or more pharmaceutically acceptable excipients.

### BRIEF DESCRIPTION OF THE DRAWINGS

FIG. 1 is an illustration of powder X-ray diffraction (“PXRD”) pattern of amorphous form of valganciclovir hydrochloride prepared according to the examples 1, 2 and 6.

FIG. 2 is an illustration of PXRD pattern of amorphous form of valganciclovir hydrochloride prepared according to the examples 3 to 5.

FIG. 3 is an illustration of PXRD pattern of amorphous form of valganciclovir hydrochloride prepared according to the example 7.

### DETAILED DESCRIPTION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Note that while the specification and claims may refer to a final product such as, for example, a tablet or other dosage form of the invention as, for example, containing particles having a certain particle size or distribution, or a certain type of, for example, a specific form of a filler, it may be difficult to tell from the final dosage form that the recitation is satisfied. However, such a recitation may be satisfied if the materials used prior to final production (in the case of a tablet for example, blending and tablet formulation), for example, meet that recitation. Indeed, as to any property or characteristic of a final product which cannot be ascertained from the dosage form directly, it is sufficient if that property resides in the components recited just prior to final production steps.

Where this document refers to a material, such as in this instance, Valganciclovir, and the unique solid forms, salts, solvates and/or optical isomers thereof by reference to patterns, spectra or other graphical data, it may do so by qualifying that they are “substantially” shown or depicted in a Figure, or by one or more data points. By “substantially” used in such a context, it will be appreciated that patterns, spectra and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art should be able to compare the figures herein with a pattern generated of an unknown form of, in this case, Valganciclovir, and confirm its identity as one of the forms disclosed and claimed herein. The same holds true for other techniques which may be reported herein as well as for distinguishing between amorphous forms.

In addition, where a reference is made to a figure, it is permissible to, and this document includes and contemplates, the selection of any number of data points illustrated in the figure which uniquely define that solid form, salt, solvate, and/or optical isomer, within any associated and recited margin of error, for purposes of identification.

A reference to a molecule such as, in this case, Valganciclovir, unless otherwise specified or inconsistent with the disclosure in general, refers to any salt, amorphous form, optical isomer and/or solvate form thereof.

When a molecule or other material is identified herein as “pure”, it generally means, unless specified otherwise, that the material is about 99.0% pure or more. In general, this refers to purity with regard to unwanted residual solvents, reaction byproducts, impurities and unreacted starting materials. In the case of solid forms such as amorphous, “pure” also means 99.0% of one amorphous form free from crystalline forms, as appropriate. “Substantially” pure means, the same as “pure” except that the lower limit is about 98.0% pure or more and likewise, “essentially” pure means the same as “pure” except that the lower limit is about 95.0% pure.

It has been the endeavor of the inventors to provide processes for the preparation of amorphous forms of drug substances, more specifically, thermodynamically stable forms of drug substances, which would have the strengths of the crystalline forms, viz. thermodynamic stability, and those of the amorphous form, viz. enhanced solubility, rapid onset of action and an enhanced bioavailability. The amorphous forms are also contemplated.

In one aspect, the present patent application provides essentially pure, substantially pure, and/or pure amorphous valganciclovir hydrochloride with reference to crystalline forms. Pure amorphous form of the present invention may also have a greater polymorphic purity as measured by XRD of greater than about 99.0% or greater than about 99.5% or greater than about 99.7%.

The amorphous form of the present invention may have a chemical purity by High Performance Liquid Chromatography (HPLC) of greater than about 94.0% or greater than about 95.0% or greater than about 96.0% or greater than about 97.0%.

In an embodiment, the present application provides a process for the preparation of amorphous valganciclovir hydrochloride; which includes:

(a) providing a solution, suspension or dispersion including valganciclovir hydrochloride, either alone or in combination with one or more pharmaceutically acceptable carriers in a solvent; and

(b) removing the solvent(s) to provide the desired amorphous valganciclovir hydrochloride.

Note that, for example only, the active can be in solution while the carrier could be dispersed. Again, for example only, they could all be dissolved as well.

step a) involves providing a solution, suspension or dispersion of valganciclovir hydrochloride, either alone or in combination with one or more pharmaceutically acceptable carriers in a solvent:

The solution, suspension or dispersion of valganciclovir hydrochloride in step (a) may be provided either by dissolving valganciclovir hydrochloride in a suitable organic solvent(s) or it may be provided directly from a reaction mixture containing it that is obtained during the course of its manufacture. Suitable solvent that may be used in step a) may be selected from water; various classes of solvents, such as for example, alcoholic solvents, ketones, esters, ethers, halogenated solvents, hydrocarbons, nitriles, water aprotic polar solvents or mixtures thereof. Alcohol solvents such as for example methanol, ethanol, denatured spirit, n-propanol, isopropanol, n-butanol, isobutanol, and t-butanol; ketonic solvents such as acetone, propanone, and 2-butanone; halogenated solvents, such as dichloromethane, 1,2-dichloroethane, chloroform, and carbontetrachloride; ester solvents, such as ethyl acetate, n-propyl acetate, isopropylacetate and n-butyl acetate and the like; ether solvents such as for example dimethylether, diethylether, methyltertiarybutylether, ethylmethylether, diisopropylether, tetrahydrofuran, and dioxane. The hydrocarbon may be any solvent from this class such as for example toluene, xylene, cyclohexane, n-hexane, and n-heptane. The nitrile solvents may include acetonitrile, and propionitrile; aprotic polar solvents, such as N, N-dimethylformide (DMF), Dimethylsulfoxide (DMSO), and N, N-dimethylacetamide (DMA) or mixtures thereof.

When amorphous valganciclovir hydrochloride is prepared along with one or more pharmaceutically acceptable carriers, optionally, one or more pharmaceutically acceptable carriers may be added to a reaction mixture containing valganciclovir hydrochloride or valganciclovir hydrochloride and one or more pharmaceutically acceptable carriers may be dissolved, dispersed or suspended in the said mixture(s) or the mixtures obtained separately by dissolving valganciclovir hydrochloride and one or more pharmaceutically acceptable carriers may be combined; for providing the solution, suspension or dispersion in step a). The solvent(s) for providing the solution of valganciclovir hydrochloride along with one or more pharmaceutically acceptable carriers in step a) may be selected from the above list mentioned for providing solution.

Suitable pharmaceutically acceptable carriers that may be used in combination with any form of valganciclovir hydrochloride include but are not limited to: hydrophilic carriers like polymers of N-vinyl pyrrolidone commonly known as polyvinylpyrrolidine (“PVP” or “povidone”), gums, cellulose derivatives, cyclodextrins, gelatins, hypromellose phthalate, sugars, polyhydric alcohols, polyethylene glycol, polyethylene oxides, polyoxyalkylene derivatives, methacrylic acid copolymers, polyvinyl alcohol, and propylene glycol derivatives.

Useful pyrrolidones are homopolymers or copolymers of N-vinyl pyrrolidone. Such polymers are known to form complexes with a variety of compounds. The water-soluble forms of N-vinyl pyrrolidone are available in a variety of viscosity and molecular weight grades and may be chosen from but not limited to PVP K-12, PVP K-15, PVP K-17, PVP K-25, PVP K-30, PVP K-90, PVP K-120 and the like. Any of the above mentioned pharmaceutically acceptable carriers could be chosen or their mixtures or their mixtures with any of the excipients mentioned above.

Any pharmaceutical carrier is acceptable as long as it allows the formation of the amorphous valganciclovir hydrochloride it is compatible with the valganciclovir hydrochloride and is acceptable for human use. The choice of such a carrier is within the scope of understanding of a person skilled in the art and is not limited by the list of polymers and excipients listed above.

The dissolution temperature for providing the solution, suspension or dispersion of valganciclovir hydrochloride, optionally along with one or more pharmaceutically acceptable carriers may be less than about 130° C. or less than about 100° C. or less than about 80° C. or less than about 60° C. or less than about 40° C. or reflux temperature of the solvents used or any other suitable temperature.

Step b) involves removing the solvent(s) from the solution, suspension or dispersion obtained in step a) to provide the desired amorphous valganciclovir hydrochloride:

The solvent(s) may be removed from the solution, suspension or dispersion by techniques known in art which includes but are not limited to: distillation, evaporation, oven drying, tray drying, rotational drying (such as the Buchi Rotavapor), spray drying, freeze-drying, fluid bed drying, flash drying, spin flash drying and Ultrafilm agitated thin film dryer-vertical (ATFD-V) and the like.

BUCHI Mini Spray Dryer B-2901 BUCHI Inert Loop B-295 may be used for carrying out spray drying operations.

The solvent(s) may be removed from the solution, suspension or dispersion optionally under reduced pressure of less than about 100 mbar or less than about 60 mbar or less than about 30 mbar or less than about 10 mbar or less than about 1 mbar or any other suitable pressure. Suitable temperature that may be used for said removal of solvent(s) may be less than about 125° C. or less than about 100° C. or less than about 80° C. or less than about 60° C. or less than about 40° C. or any other suitable temperature.

The resulting product after removal of solvent(s) in step (b) may be optionally further dried in conventional manner optionally under reduced pressure. Drying temperature may about 125° C. or less than about 100° C. or less than about 80° C. or less than about 60° C. or less than about 40° C. or any other suitable temperature and in presence or absence of inert atmosphere. Other conventional drying methods known in the art may also be used.

Valganciclovir hydrochloride that is used as the input in the process of the present application may be of any form known in the art. Any form of valganciclovir or its salt form or its precursor intermediate can also be used as starting material for the preparation of amorphous valganciclovir hydrochloride by following the processes described above or the processes known to a person skilled in the art.

if desired, the amorphous form of valganciclovir hydrochloride or its combination with one or more pharmaceutically acceptable carriers thus obtained form step b) may be subjected to particle size reduction by ball milling, roller milling, micronizing, hammer milling, jet milling, grinding and the like to get the desired particle size.

Specifically contemplated are pharmaceutical compositions that include amorphous valganciclovir hydrochloride and at least one pharmaceutically acceptable carrier.

Amorphous valganciclovir hydrochloride may be formulated as solid compositions for oral administration in the form of capsules, tablets, pills, powders or granules. In an embodiment of the present invention, the active product in the compositions is mixed with one or more pharmaceutically acceptable excipients. The drug substance may be formulated as liquid compositions for oral administration including for example solutions, suspensions, syrups, elixirs and emulsions, containing solvents or vehicles such as water, sorbitol, glycerine, propylene glycol or liquid paraffin, may be used.

The compositions for parenteral administration can be suspensions, emulsions or aqueous or non-aqueous, sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable carriers include, but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methyl cellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, complex forming agents such as various grades of cyclodextrins, resins; release rate controlling agents such as hydroxypropyl cellulose, hydroxymethyl cellulose, hydroxypropyl methyl cellulose, ethyl cellulose, methyl cellulose, various grades of methyl methacrylates, waxes and the like. Other pharmaceutically acceptable excipients that are of use include but not limited to film formers, plasticizers, colorants, flavoring agents, sweeteners, viscosity enhancers, preservatives, antioxidants and the like.

In a further embodiment the processes of the present invention produce the desired amorphous valganciclovir hydrochloride with high yield and purity.

X-Ray powder diffraction pattern of amorphous valganciclovir hydrochloride of the present application may be measured by using Cu K∝ radiation, having the wavelength 1.5418 Å, and the results reported herein were obtained using a Bruker AXS D8 Advance Powder X-ray Diffractometer.

Chemical purity of amorphous valganciclovir hydrochloride of the present application may be measured by HPLC method that is disclosed in US pharmacopoeial forum 32(2) [March-April 2006].

Having described the invention with reference to certain embodiments, other embodiments will become apparent to one skilled in the art from consideration of the specification. The invention is further defined by reference to the following examples describing the preparation of the amorphous valganciclovir hydrochloride. It will be apparent to those skilled in the art that many modifications, both to materials and methods, may be practiced without departing from the scope of the invention.

### EXAMPLES

### Example 1

### A Process for the Preparation of Amorphous Valganciclovir Hydrochloride Using Methanol

Valganciclovir hydrochloride (5.0 g) was dissolved in methanol (35 ml) at about 40° C. to about 45° C. and the solution was filtered to remove any undissolved particle. The clear solution was spray dried at about 75° C., 5.0-kg/cm2 nitrogen pressure, aspirator at 70% (volume flow=28m3/h) and at a rate of about 6.0 ml per minute. A spray dryer was operated under closed loop nitrogen circulation with nitrogen as the drying and spraying medium with oxygen content less than 6% in the inert loop. The material was recovered from cyclone chamber. Yield: 3.0 g.

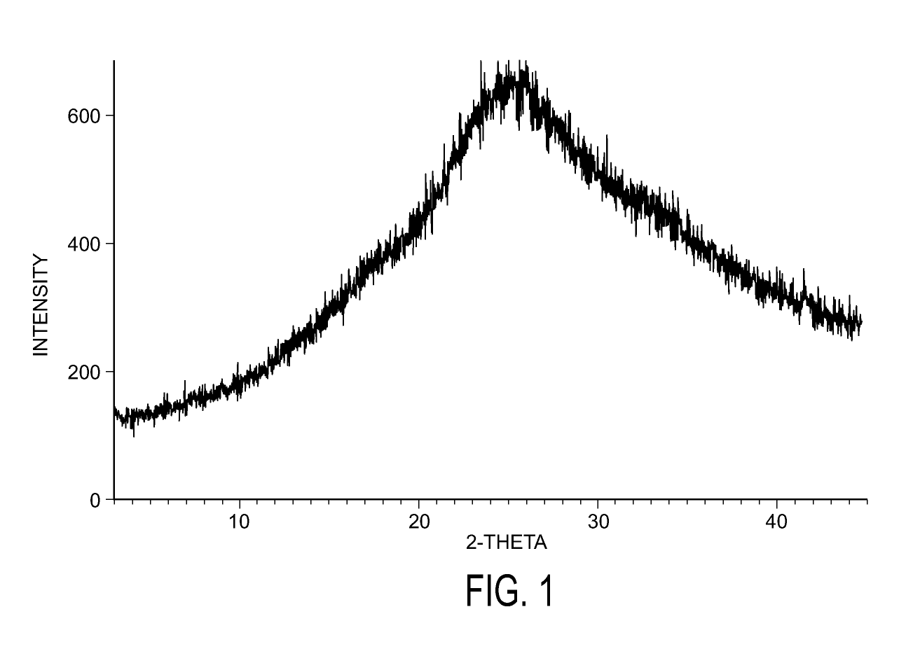

The PXRD pattern of amorphous form of valganciclovir hydrochloride may be illustrated by FIG. 1.

### Example 2

### A Process for the Preparation of Amorphous Valganciclovir Hydrochloride Using Water

Valganciclovir hydrochloride (5.0 g) was dissolved in water (24 ml) at about 50° C. to about 55° C. and the solution was filtered to remove any undissolved particle. The clear solution was spray dried at about 100° C., 5.0-kg/cm2 nitrogen pressure, aspirator at 70% (volume flow=28m3/h) and at a rate of about 1.5 ml per minute. A spray dryer was operated under closed loop nitrogen circulation with nitrogen as the drying and spraying medium with oxygen content less than 6% in the inert loop. The material was recovered from cyclone chamber. Yield: 2.2 g.

The PXRD pattern of amorphous form of valganciclovir hydrochloride may be illustrated by FIG. 1.

### Example 3

### A Process for the Preparation of Valganciclovir Hydrochloride Amorphous Solid Dispersion With Povidone K-30

Valganciclovir hydrochloride (2.0 g) and povidone (K-30) (2.0 g) were dissolved in methanol (30 ml). The resulting solution was distilled completely at about 65° C. to about 70° C. under vacuum to afford 3.4 g of valganciclovir hydrochloride amorphous solid dispersion with povidone. Yield: 3.4 g.

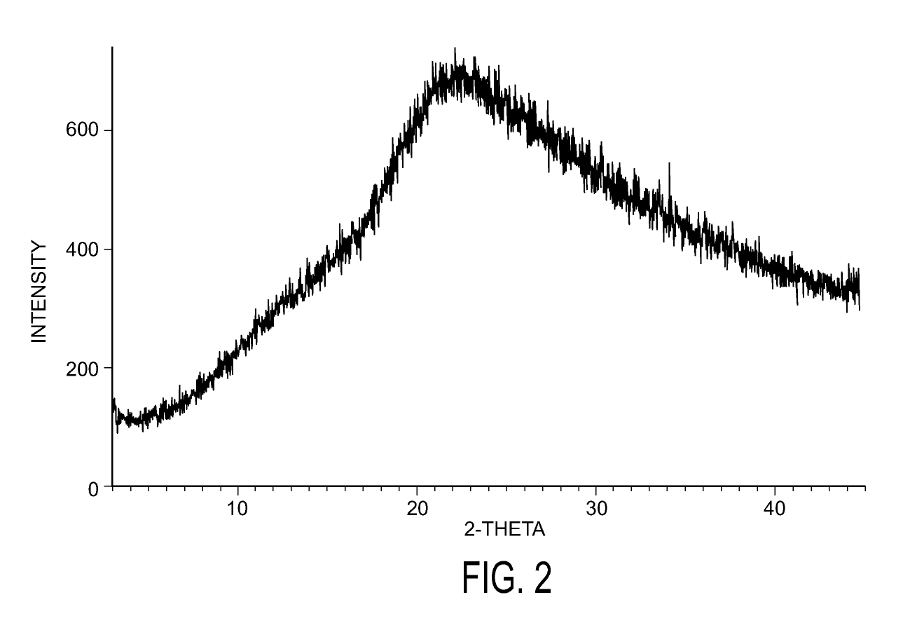

The PXRD pattern of amorphous form of valganciclovir hydrochloride may be illustrated by FIG. 2.

### Example 4

### A Process for the Preparation of Valganciclovir Hydrochloride Amorphous Solid Dispersion With HPMC

Valganciclovir hydrochloride (2.0 g) and hydroxy propyl methyl cellulose (HPMC) (2.0 g) were dissolved in methanol (30 ml). The resulting solution was distilled completely at about 65° C. to about 70° C. under vacuum to afford valganciclovir hydrochloride amorphous solid dispersion with hydroxy propyl methyl cellulose. Yield: 3.4 g.

The PXRD pattern of amorphous form of valganciclovir hydrochloride may be illustrated by FIG. 2.

### Example 5

### A Process for the Preparation of Valganciclovir Hydrochloride Amorphous Solid Dispersion With Ethyl Cellulose

Valganciclovir hydrochloride (2.0 g) and ethyl cellulose (2.0 g) were dissolved in methanol (30 ml). The resulting solution was distilled completely at about 65° C. to about 70° C. under vacuum to afford valganciclovir hydrochloride amorphous solid dispersion with ethyl cellulose. Yield: 3.6 g.

The PXRD pattern of amorphous form of valganciclovir hydrochloride may be illustrated by FIG. 2.

### Example 6

### A Process for the Preparation of Amorphous Valganciclovir Hydrochloride Using Methanol in Buchi Rotavapour

Valganciclovir hydrochloride (2.0 g) was dissolved in methanol (20 ml) at about 25° C. to about 30° C. and the solution was distilled completely under vacuum at about 65° C. in a Buchi rotavapour. The resulting solid was dried in Buchi rotavapour for about 2 hours under vacuum at 65° C. to afford amorphous Valganciclovir hydrochloride. Yield: 1.7 g

The PXRD pattern of amorphous form of valganciclovir hydrochloride may be illustrated by FIG. 1.

### Example 7

### A Process for the Preparation of Amorphous Valganciclovir Hydrochloride Using Methanol

Valganciclovir hydrochloride (30.0 g) was dissolved in methanol (140 ml) at about 40° C. to about 45° C. The solution was filtered to remove any undissolved particle and washed with methanol (70 ml). The clear solution was spray dried at about 75° C., 5.0-kg/cm2 nitrogen pressure, aspirator at 70% (volume flow=28m3/h) and at a rate of about 6.0 ml per minute. A spray dryer was operated under closed loop nitrogen circulation with nitrogen as the drying and spraying medium with oxygen content less than 6% in the inert loop. The material was recovered from cyclone chamber. Yield: 18.4 g (degree of crystallinity: less than 0.3%).

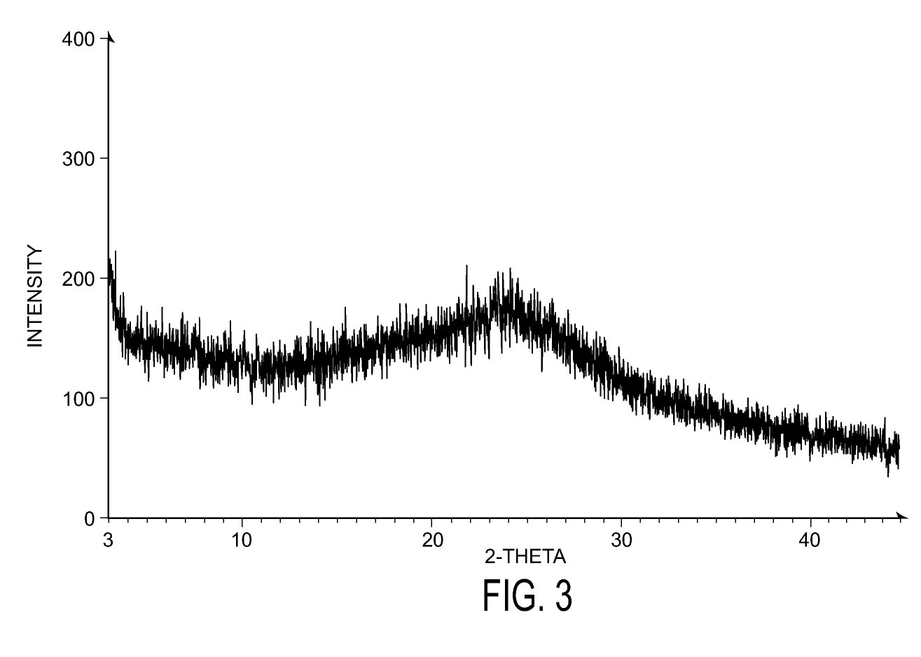

The PXRD pattern of amorphous form of valganciclovir hydrochloride may be illustrated by FIG. 3.

1. (canceled)

2. A process for preparation of amorphous valganciclovir hydrochloride, which includes:
a) providing a solution, suspension or dispersion of valganciclovir hydrochloride, either alone or in combination with one or more pharmaceutically acceptable carriers, in a solvent; and
b) removing solvent from the solution to provide the desired amorphous valganciclovir hydrochloride.

3. The process of claim 2 wherein the solvent is water or one or more organic solvents or mixtures thereof.

4. The process of claim 2 wherein the solvent is removed in step b) by distillation, evaporation, oven drying, tray drying, rotational drying, spray drying, freeze-drying, fluid bed drying, flash drying, spin flash drying or thin film drying.

5. The process of claim 2 wherein the solvent is removed in step b) by spray drying.

6. The process of claim 2 wherein the solvent is removed in step b) by spray drying using BUCHI Mini Spray Dryer B-290/BUCHI Inert Loop B-295.

7. The process of claim 5 wherein the spray dryer comprises a single or two fluid nozzles.

8. Amorphous Valganciclovir hydrochloride having a polymorphic purity of greater than 99.0% by powder X-ray diffraction.

9. Amorphous Valganciclovir hydrochloride having a polymorphic purity of greater than 99.5% by powder X-ray diffraction.

10. The process of claim 2, wherein in step a) valganciclovir hydrochloride is dissolved in a solvent comprising an alcohol.

11. The process of claim 2, wherein in step a) valganciclovir hydrochloride is dissolved in a solvent comprising methanol.

12. The process of claim 2, wherein in step a) valganciclovir hydrochloride is dissolved in a solvent comprising water.

13. The process of claim 2, wherein in step a) valganciclovir hydrochloride and a polymer are dissolved in a solvent comprising an alcohol.

14. The process of claim 2, wherein in step a) valganciclovir hydrochloride and a polymer are dissolved in a solvent comprising methanol.

15. The process of claim 13, wherein a polymer comprises a N-vinylpyrrolidone polymer.

16. The process of claim 13, wherein a polymer comprises a cellulose derivative.

17. The process of claim 13, wherein a polymer comprises a hydroxypropyl methylcellulose.

18. The process of claim 6 wherein the spray dryer comprises a single or two fluid nozzles.

19. A process for preparing amorphous valganciclovir hydrochloride, comprising:
a) providing a solution of valganciclovir hydrochloride in a solvent comprising an alcohol, optionally in combination with one or more pharmaceutically acceptable polymers; and
b) removing solvent to form a solid, using thin film drying.

20. The process of claim 19, wherein a solvent is methanol.

21. The process of claim 19, wherein a pharmaceutically acceptable polymer comprises a N-vinylpyrrolidone polymer or a cellulose derivative.
